(19) United States
(12) Patent Application Publication
(10) Pub. No.: US 2024/0009127 A1
(43) Pub. Date: Jan. 11, 2024

(54) METHODS AND COMPOSITIONS FOR NUTRITIONAL SUSTENANCE AND WASTE SCAVENGING IN TISSUE INJURIES AND WOUNDS

(71) Applicant: New York Society for the Relief of the Ruptured and Crippled, Maintaining the Hospital For Special, New York, NY (US)

(72) Inventors: Guang-Ting Cong, New York, NY (US); Matthew Cunningham, New York, NY (US)

(21) Appl. No.: 18/250,459

(22) PCT Filed: Oct. 27, 2021
(86) PCT No.: PCT/US2021/056763

§ 371 Date: Apr. 25, 2023

Related U.S. Application Data
(60) Provisional application No. 63/106,745, filed on Oct. 28, 2020.

(51) Int. Cl. A61K 9/16; A61K 31/198; A61K 31/4172; A61K 31/405; A61K 31/14; A61K 31/197; A61K 31/519; A61K 31/455; A61K 31/4415; A61K 31/51; A61K 31/19; A61K 9/50; A61P 17/02; A61L 27/54; A61L 27/56

(52) U.S. Cl. CPC A61K 9/1647; A61K 31/198; A61K 31/4172; A61K 31/405; A61K 31/14; A61K 31/197; A61K 31/519; A61K 31/455; A61K 31/4415; A61K 31/51; A61K 31/19; A61K 9/1611; A61K 9/5015; A61P 17/02; A61L 27/54; A61L 27/56; A61L 2430/02; A61L 2300/622; A61L 2300/404

(57) ABSTRACT

Methods and compositions are described for the introduction of nutritional support material and optional waste scavenging material at the site of tissue injury, disease, surgery, or wound. These methods and compositions are useful for the purpose of optimizing the tissue healing environment at the site of tissue injury, disease, surgery, or wound.

### BACKGROUND OF THE INVENTION

Sites of tissue injury, disease, surgery, or wounds, such as in surgical incisions and dissection, fracture, tendon and ligamentous tears, spinal fusion, joint reconstruction, trauma, fracture or other physiological circumstances requiring subsequent tissue healing, are acutely injured by the original trauma, disease or injury, as well as by the processes of surgery, such as periosteal stripping and use of electrocautery. As a result, tissue damage caused by injury and surgery can significantly negatively affect tissue healing. Furthermore, injury and surgery often occur at anatomic sites where the biological milieu is immunoprivileged and undervascularized, such as in and surrounding articular joints and the intervertebral disc space, which additionally delay natural tissue healing. Postoperatively, neovascularization and ingrowth of tissue healing can take weeks to occur, during which exists a non-optimal host environment for tissue survival.

Tissue healing at sites of tissue injury, disease, surgery, or wound typically occur via biologic processes intrinsic to the host organism, including formation of a hematoma leading to infiltration of immune cells, eventually causing formation of robust neovascularization via granulation tissue and callus. These intrinsic processes can take days to weeks to establish a biologic milieu that can consistently sustain tissue homeostasis. Extrinsic contributions to healing are provisioned through such interventions as surgery for fracture stabilization, plastic reconstruction to augment soft tissue defects, or bone and cellular grafting by either surgical implantation or injection to provide additional biologic supplementation. A commonly used method to improve bone healing, for example, is the surgical implantation of autologous bone graft at sites that require biological bony supplementation. Grafted cells have been shown to be intricately involved in contributing to the effectiveness of autologous bone grafts. However, autologous bone marrow aspirates or bone graft obtained from long bones and iliac crest can contain as little as 0.01% to 0.001% mesenchymal stem cells—cells which are key to successful tissue healing.

Poor cellular- or bone-graft handling intra-operatively, and implantation into atrophic/biology-poor host sites prior to complete establishment of intrinsic biologic support, can lead to loss of stem cells and graft death, and thus lead to imperfect healing rates such as those seen in cell-augmented orthopedic long-bone and spine surgery as well as other tissue healing and tissue repair situations. This situation frequently leads to decreased rates of graft survival, bone healing, and associated rates of tissue repair.

There exists a need in the art for methods and compositions that encourage cellular healing, survival, and repair at the site of tissue injury, disease, surgery, wound and/or other tissue defects, that can sustain tissue homeostasis until intrinsic biologic processes can assume the same role.

### SUMMARY OF THE INVENTION

This invention is based upon the inventors' theory that poor cellular viability, and/or the inability to trigger a regenerative phenotype due to lack of local tissue homeostasis, contribute to imperfect healing rates seen in cell-augmented orthopedic long-bone and spine surgery as well as other tissue healing situations.

In one aspect, a nutritional support material for supplementing tissue survival is provided. The material includes a) a slow-elution carrier for controlling nutrient release; and b) a nutrient cocktail that comprises glucose; and wherein the slow-elution carrier has a release half-life of between 30 minutes and 14 days. In certain embodiments, the nutritional support material is implantable or injectable. In certain embodiments, the nutrient cocktail contains a powdered commercial cell medium, such as DMEM, EMEM, RPMI, IMDM, Ham's F10, or Ham's F12 media powder. In certain embodiments, the material is in the form of a microparticle between 1 micron and 5 mm in diameter. In certain embodiments, the carrier is a carrier polymeric matrix, optionally PLGA.

In another aspect, a method of supplementing tissue survival relating to injury, disease, or surgery is provided. The method includes administering the nutritional support material as described herein to the site of tissue injury, disease, surgery, or wound. In certain embodiments, the method further includes administering a waste scavenging material at the site of the tissue injury, disease, surgery, or wound. In certain embodiments, the method includes administering the nutritional support material, optionally in combination with the waste scavenging material, with a bone graft. In other embodiments, the method includes administering the nutritional support material, optionally in combination with the waste scavenging material, with a cell therapy.

In one aspect, a method of increasing tissue healing at the site of tissue injury, disease, surgery, or wound comprises introducing to a subject in need thereof a nutritional support material at the site of the tissue injury, disease, surgery, or wound to improve tissue survival in the injury environment. In one embodiment, the material is introduced during a surgical procedure prior to closure of the site of tissue injury, disease or wound.

In another aspect, a method of increasing tissue healing at the site of tissue injury, disease, surgery, or wound comprises introducing to a subject in need thereof a waste scavenging material at the site of the tissue injury, disease, surgery, or wound to improve cell survival in the injury environment. In one embodiment, the material is introduced during a surgical procedure prior to closure of the site of tissue injury, disease or wound.

In still another aspect, a method of increasing tissue healing at the site of tissue injury, disease, surgery, or wound comprises introducing to a subject in need thereof a nutritional support material and a waste scavenging material at the site of the tissue injury, site of surgery, or wound to improve cell survival in the injury environment. In one embodiment, the material is introduced during a surgical procedure prior to closure of the site of tissue injury, disease, or wound.

In still another aspect, a composition for increasing healing at the site of tissue injury, disease, surgery, or wound comprises an effective amount of a nutritional support material in a physiologically acceptable delivery system suitable to delivery to the site of the tissue injury, disease, surgery, or wound.

In still another aspect, a composition for increasing healing at the site of tissue injury, disease, surgery, or wound comprises an effective amount of a waste scavenging material in a physiologically acceptable delivery system suitable to delivery to the site of the tissue injury, disease, surgery, or wound.

In another aspect, a composition for increasing healing at the site of a tissue injury, disease, surgery, or wound comprises an effective amount of a nutritional support material and waste scavenging material in a physiologically acceptable delivery system suitable to delivery to the site of tissue injury, disease, surgery or wound.

Yet another aspect is a composition for implantation into the site of tissue injury, disease, surgery, or wound that is coated or impregnated with a nutritional support material and/or waste scavenging material which is capable of its intended effect at the site of said implantation.

Still other aspects and advantages of these compositions and methods are described further in the following detailed description of the preferred embodiments thereof.

### BRIEF DESCRIPTION OF THE DRAWINGS

FIGS. 1A-1C. An example of a fabrication method for Dulbecco's Modified Eagle Medium (DMEM)-loaded nutrient poly(lactic-co-glycolic acid) (PLGA) microparticles. FIG. 1A. Stepwise diagram showing formation of the primary emulsion, its dropwise addition to 0.5% polyvinyl alcohol (PVA) under vortex to form the secondary emulsion, and final washing to obtain hardened microparticles. FIG. 1B. Hardened microparticles settle by gravity after completion of the secondary emulsion step. FIG. 1C. 40× magnification darkfield microscopy of microparticles demonstrating coloration resulting from DMEM powder embedded within PLGA spheres, in contrast to native PLGA which is white in color.

FIGS. 2A-2C. Experimental data demonstrating necessity of essential nutrients for cell survival of human bone marrow mesenchymal stem cell (hBM MSC). Particles were fabricated using PLGA loaded with Dulbecco's Modified Eagle Medium (DMEM)—a standardized cell culture medium nutritional composition. FIG. 2A. Timepoint viability was performed using hBM MSCs in buffered saline supplemented with nutrient particles versus without. At 10 hours, light microscopy demonstrated healthy viable cells in presence of nutrient particles (FIG. 2B), versus near-complete cell death in absence of particles (FIG. 2C).

FIG. 3. An example embodiment of a protocol for in vitro testing of cell viability with versus without microparticles placed in well inserts that release nutrition and/or scavenge waste.

FIG. 4. An example embodiment of the introduction of nutritional support and/or waste disposal materials, in this case microparticles, into the site of tissue injury or wound, for the purposes of improving tissue healing.

FIG. 5. Graph showing the elution profile of a DMEM-based wax-coated solid core microparticle demonstrating slow-release profile with desirable initial burst release.

FIG. 6. Using primary bone marrow-derived human mesenchymal stem cells (hBM-MSCs) as a model cell type, in vitro viability of these cell types can be sustained using controlled-release nutrient microparticles. Synergistic effect was seen when used in conjunction with fetal bovine serum (FBS) in culture.

FIG. 7. Though charcoal-based waste-scavenging microparticles demonstrated minimal cell viability effect on its own, it demonstrated synergistic effect in promoting cell viability in vitro when used in conjunction with controlled-release nutrient microparticles.

FIG. 8. Differentiation assay of 3-week survived human bone marrow mesenchymal stem cells (hBM-MSCs) demonstrate maintenance of stem cell pluripotency (adipogenic, chondrogenic, and osteogenic differentiation) when co-cultured with nutrient-eluting microparticles, to a degree that is at least equivalent to 10% fetal bovine serum (FBS)-survived hBM-MSCs (positive control). This suggests that nutrient supplementation may improve For comparison, undifferentiated hBM-MSCs (negative control) demonstrate low levels of staining for differentiation markers. Upper panels demonstrate qualitative Oil Red-O, Safranin-O, and Alizarin Red monolayer staining for detection of adipogenic, chondrogenic, and osteogenic differentiation, respectively. Lower panel demonstrates absorbance quantification of retained stain.

FIG. 9. Radiographic evidence in a rat posterolateral lumbar spinal fusion model that provision of nutrient microparticles alongside iliac bone autograft improves bony fusion at 8 weeks (A) as compared to using blank particles (B). White arrows refer to surgical level L4-L5.

### DETAILED DESCRIPTION

The presently described methods and compositions address this need in the art by introducing nutritional support and/or toxin disposal materials into injury, surgery and/or other wound sites to improve local cell and tissue survival. Localized nutritional supplementation and waste scavenging by direct implantation or injection to an injury or surgery site is a novel technique in supporting viability and healing of damaged tissue, and can have significant impact on the future practice of surgery as well as in other domains of medical practice.

These methods and compositions specifically provide potential for human application in surgery for improving bone and soft tissue healing. There is direct and inherent bench to bedside relevance in the area of orthopedic surgery, especially in spine, reconstructive joint, and trauma applications, where surgeons often encounter devitalized tissue defects and face the difficulty of regenerating tissue in poor biological environments. These methods and compositions are intimately relevant to improving function, eliminating pain, and restoring mobility in spine, trauma, and reconstructive orthopedics due to a focus on improving bone grafting and bone healing. This invention has broad potential applications in, but not limited to, craniofacial reconstruction, fracture healing, spinal fusion, orthopedic soft tissue healing (tendon, muscle, ligament, intervertebral disc), surgical wound healing, traumatic wound healing, and visceral organ healing. In addition to uses with bone grafting, the compositions and methods of this technology are useful with microparticle implantation, injection, spray, coating on an implant, en bloc implantation, etc.

These methods and compositions for stimulating tissue healing through provision of nutrition and waste disposal, in some embodiments, obviate the need for biologically active drugs and molecules that, when used medically or surgically in non-physiological doses, have the potential to cause harm.

Thus, in one aspect, described in detail below, a composition is provided for increasing healing at the site of a bone defect, injury or wound when placed in the direct site of the injury, defect or wound. In one aspect, the composition comprises an effective amount of a nutritional support material in a physiologically acceptable delivery system suitable for direct delivery to the site of the defect, injury or wound. In another aspect the composition comprises an effective amount of a waste disposal material in a physiologically acceptable delivery system for direct delivery to the site of the defect, injury or wound. In another aspect the composition comprises an effective amount of nutritional support material and a waste disposal material in a physiologically acceptable delivery system for direct delivery to the site of the defect, injury or wound.

In another aspect, as described in detail below, method of increasing healing of a tissue defect, graft, injury, surgical site, or wound comprises introducing nutritional support material and/or waste disposal materials at the site of tissue defect, graft, injury, surgical site, or wound to improve cell survival in the wound environment.

### I. Components and Definitions Useful in the Methods and Compositions

A. Nutrition and Nutritional Supplement Materials

Amongst medical practitioners, it is agreed that nutrition is of high importance in the repair, regeneration, and healing of injured tissues. Proper dietary nutrition is known to improve tissue healing, in part evidenced by the fact that in extensive traumatic or burn injuries there can be a many-fold increase in the body's daily caloric requirements from the resulting healing response. Further evidence exists in cases of impaired kidney or liver function or vascular disease, which cause electrolyte imbalances and poor tissue nutrition delivery, and thus an associated poor wound healing response. The importance of cellular nutrition crosses medical and surgical disciplines; amongst orthopedic surgeons, for example, it is agreed that bony fractures that occur in a patient who is severely emaciated from malignancy or end-stage multiorgan disease or starvation essentially do not heal. Plastic surgeons often stress the importance of nutrition in the management of wounds and chronic ulcers. Vascular surgeons who manage patients with amputations and limb gangrene have shown improvement in healing when nutritional profiles are optimized perioperatively, and nutritional status has been shown as a predictor for decreased surgical risk and improved outcomes.

The inventors demonstrate that tissue healing, whether as a response to a surgical intervention, injury, or disease, also benefits from direct nutritional supplementation to the affected site. A surgical or injured tissue bed, however, consists of a degree of local tissue devitalization, which is not conducive to intrinsic nutrient delivery from the host organism. Thus, the methods and compositions described herein provide nutrition to injured tissue via a process of direct application/implantation, with the purpose of improving healing outcomes across medical and surgical disciplines.

The “nutritional support material” provided herein comprises a “nutrient cocktail”. The “nutrient cocktail” as used herein means a composition that contains one or a mixture of multiple sugars, amino acids, vitamins, fatty acids, minerals, salts, and nucleic acids that maintain a physiological pH. In certain embodiments, the nutrient cocktail contains, at a minimum, glucose. In certain embodiments, the nutrient cocktail can comprise pH-buffering salts, sodium and potassium salts, glucose, thiamine, riboflavin, niacin, pantothenic acid, pyridoxine, biotin, folates, cobalamin, vitamin A, vitamin D, vitamin E, vitamin K, vitamin C, alanine, arginine, asparagine, aspartic acid, cysteine, glutamine, glutamic acid, glycine, glycine, histidine, isoleucine, leucine, lysine, methionine, phenylalanine, proline, serine, threonine, tryptophan, tyrosine, valine, taurine, glucuronolactone, carnitine, creatine, sodium pyruvate, adenine, adenosine, guanine, cytosine, thymine, uracil, dietary fatty acids (saturated, monounsaturated, and polyunsaturated fatty acids of 4 to 24 carbons in length such as palmitic acid, stearic acid, oleic acid, linoleic acid, linolenic acid, arachidonic acid, eicosapentanoic acid, docosahexanoic acid, omega-3 fatty acids, and omega-6 fatty acids), cholesterol, magnesium, potassium, calcium, boron, zinc, copper, iron, phosphorous, vanadium, strontium, silicon, selenium, nickel, or manganese. In another embodiment the nutrient cocktail can include all essential amino acids, sugars, vitamins, nucleic acids, and buffering salts necessary for the maintenance of tissue homeostasis.

In another embodiment, the nutrient cocktail is derived from a cell culture medium formulation, such as that of the ATCC 30-2002 formulation for Dulbecco's Modified Eagle Medium (DMEM) including the inorganic salts CaCl₂, Fe(NO₃)₃·9H₂O, MgSO₄, KCl, NaHCO₃ NaCl, NaH₂PO₄·H₂O and/or the amino acids L-arginine·HCl, L-cystine·2HCl, L-glutamine, glycine, L-histidine·HCl·H₂O, L-isoleucine, L-leucine, L-lysine·HCl, L-methionine, l-Phenylalanine, L-serine, L-threonine, L-tryptophan, L-tyrosine·2Na·2H₂O, L-valine, and/or vitamins such as choline chloride, folic acid, myo-inositol nicotinamide, d-pantothenic acid, (hemicalcium) pyridoxine·HCl, riboflavin, thiamine·HCl. Still other components include d-glucose, phenol red, sodium salt, sodium pyruvate. Other nutritional components can include the components of other known culture medium formulations such as RPMI 1640, EMEM, or other cell culture formulation variants thereof. Various mixtures of the components of known cell culture media can be introduced into a nutritional materials mixture. However, well known cell culture medium is a convenient mixture, known to be safe and efficacious for in vitro cell growth. Some commonly used cell culture media are described herein. These media are available in powdered form from Fisher Scientific and other suppliers. In certain embodiments, the nutrient cocktail is provided as a powdered cell culture medium. In other embodiments, the nutrient cocktail is provided as a liquid or semi-solid cell culture medium.

Eagle's Minimum Essential Medium (EMEM) was among the first widely used media and was formulated by Harry Eagle from a simpler basal medium (BME). EMEM contains balanced salt solution, nonessential amino acids, and sodium pyruvate.

Dulbecco's Modified Eagle's Medium (DMEM) has almost twice the concentration of amino acids and four times the amount of vitamins as EMEM, as well as ferric nitrate, sodium pyruvate, and some supplementary amino acids. The original formulation contained 1,000 mg/L of glucose and was first reported for culturing embryonic mouse cells. A further variation with 4500 mg/L of glucose has been proved to be optimal for the culture of various types of cells. DMEM is a basal medium and contains no proteins or growth promoting agents. Therefore, it requires supplementation to be a “complete” medium. It is most commonly supplemented with 5-10% Fetal Bovine Serum (FBS).

RPMI-1640 is a general purpose media with a broad range of applications for mammalian cells, especially hematopoietic cells. RPMI-1640 was developed at Roswell Park Memorial Institute (RPMI) in Buffalo, New York. RPMI-1640 is a modification of McCoy's 5A and was developed for the long-term culture of peripheral blood lymphocytes. RPMI-1640 uses a bicarbonate buffering system and differs from the most mammalian cell culture media in its typical pH 8 formulation. RPMI-1640 supports the growth of a wide variety of cells in suspension and grown as monolayers.

Ham's nutrient mixtures were originally developed to support the clonal outgrowth of Chinese hamster ovary (CHO) cells. There have been numerous modifications to the original formulation including Hams's F-12 medium, a more complex formulation than the original F-10 suitable for serum-free propagation. Mixtures were formulated for use with or without serum supplementation, depending on the type of cells being cultured.

Ham's F-10: It has been shown to support the growth of human diploid cells, for example, human fibroblast cells, and white blood cells for chromosomal analysis.

Ham's F-12: It has been shown to support the growth of primary rat hepatocytes and rat prostate epithelial cells. Ham's F-12 supplemented with 25 mM HEPES provides more optimum buffering.

Coon's modification of Ham's F-12: It consists of almost two times the amount of amino acids and pyruvate as compared to F-12 and also includes ascorbic acid. It was developed for culturing hybrid cells produced by viral fusion.

DMEM/F12: It is a mixture of DMEM and Ham's F-12 and is an extremely rich and complex medium. It supports the growth of a broad range of cell types in both serum and serum-free formulations. HEPES buffer is included in the formulation at a final concentration of 15 mM to compensate for the loss of buffering capacity incurred by eliminating serum.

Iscove's Modified Dulbecco's Medium (IMDM) is a highly enriched synthetic media well suited for rapidly proliferating, high-density cell cultures. IMDM is a modification of DMEM containing selenium, and has additional amino acids, vitamins and inorganic salts as compared to DMEM. It has potassium nitrate instead of ferric nitrate and also contains HEPES and sodium pyruvate. It was formulated for the growth of lymphocytes, for example the differentiation of monocytes into macrophages, and hybridomas.

B. Waste Removal Materials

In addition to nutrient provision, waste removal is a complimentary factor in maintaining homeostasis for cells in a poor host environment. Charcoal as one embodiment has been used extensively in medical application for drug detoxification and in scientific research for in vitro plant cell cultures. There is abundant evidence that activated charcoal has a positive effect on plant cell cultures, likely due to adsorption of inhibitory compounds in the culture medium and sequestration of toxic metabolites. However, charcoal has not been explored as an adjunct to animal cell culture or as a means of promoting in vivo cell/tissue survival for surgical applications. Prior studies demonstrate that coated activated charcoal has the benefit of selective adsorption of small molecules while minimally affecting protein and other large molecules. Activated charcoal also has high affinity for toxins and wastes normally excreted in urine, while having virtually no affinity for ions, thus avoiding unwanted adsorption of small charged nutritive particles and unwanted effect on local electrolyte balance. While dextran-coated charcoal microparticles are freely available commercially, dextran has been shown to induce a disturbance of primary and secondary hemostasis, which would contraindicate its use in certain surgical applications due to the risk of bleeding. Instead, biocompatible coatings can assist in improving the in vivo utility of waste scavenging material. In one embodiment, fabrication of biocompatible coated charcoal using a cellulose coating could overcome these limitations, as cellulose would not only decrease the porosity profile of charcoal to prevent large-molecule adsorption, but would also act as a pro-coagulant to aid in hemostasis. Thus, activated charcoal with a biocompatible cellulose coating is a waste scavenging material useful in the compositions or methods of this invention.

In still another embodiment, the waste scavenging material is a flavonoid, such as described in the art. See, e.g., Tremi, J and Smejkal, K, Flavonoids as Potent Scavengers of Hydroxyl Radicals, 2016 Comprehen. Rev., 15(4):820-8738, incorporated by reference herein.

C. Delivery Vehicles Suitable for Direct Delivery to the Site of the Tissue Injury, Disease, Surgery, or Wound

Another component of the nutritional support material or waste disposal materials is a delivery vehicle suitable for the release of the nutrient cocktail/waste scavenging materials. In one embodiment the delivery vehicle is a suitable pharmaceutical excipient, diluent, buffer, or carrier selected with regard to the intended route of administration and standard pharmaceutical practice. In one embodiment, carbonate buffers or other physiologically useful and safe buffers that can be used to adjust the nutritional support materials and/or the waste disposal materials to physiological pH for application in and around a bone injury, defect or condition are useful. In one aspect, the carrier or buffer is adjusted to a pH of about 7.4.

The delivery of the nutritional and or waste scavenging supplementation can be accomplished in the form of a solid for implant, a liquid for injection, a powder, a chip, a pellet, a microparticle, a nanoparticle, a capsule, a cream, a gel, a lotion, a paste, a coating, an impregnation of an existing implant or tissue, a combination thereof, or impregnation device containing implanted nutritional/waste scavenging materials or any biologically acceptable delivery apparatus for the nutritional supplement/waste scavenging materials. The compositions may be locally administered alone (with physiological saline, or the like as medium), or may contain, in addition to various growth factors, one species or two or more delivery vehicle species selected from scaffold, extracellular matrix protein, gelation material and thickener. For instance, when administered locally using physiological saline or the like, the nutritional components/waste scavenging components can reach the entirety of bone marrow, while when a scaffold is used, these compositions can be confined to a given region of the bone, and improve the bone substance of the required portion only.

In one embodiment, using microparticle supplementation to improve tissue healing and cell viability in human surgical applications is another delivery mechanism. For instance, the introduction of key ingredients in ensuring cellular homeostasis (specifically, nutritional supplementation and waste disposal) via microparticles improves the osteogenic response and thus improves bone healing and fusion rates in an anatomic region requiring such.

A variety of such microparticles are described in U.S. Pat. No. 10,335,498, incorporated by reference herein. In one embodiment, the delivery vehicle comprises a cationic polymer microparticle utilizing e.g., polyethylenimine (PEI), chitosan, cyclodextrin or dendrimers. In one embodiment, the delivery vehicle comprises a non-cationic polymer, e.g., dioleoylphosphatidyl ethanolamine (DOPE), cholesterol, polyamidoamine (PAMAM) or poloxamer In one embodiment, the nutritional supplement material and/or waste scavenging material, is complexed with a cationic polymer and encapsulated into microparticles, e.g., PLGA. These particles, when provided at the site of the bone defect, injury, or wound, can enter into cells for tissue engineering applications. In one embodiment, PLGA particles are employed to increase the encapsulation frequency although complex formation with Poly-L-lysine (PLL) may also increase the encapsulation efficiency. Other cationic materials, for example, N-[1-(2,3-dioleoyloxy)propel]-N,N,N-trimethylammonium (DOTMA), 3-β-[N--(N,N′-dimethylaminoethane) carbamoyl] cholesterol (DC-Chol), or cetyltrimethylammonium bromide (CTAB), may be used to make microparticles. Blends of polymers can also be used to modulate encapsulation efficiency and the nutrient elution profile.

In one embodiment, a delivery vehicle is non-biologically active or minimally biologically active. One embodiment comprises inorganic microparticles, e.g., calcium phosphate or silica particles; polymers including but not limited to PLGA, polylactic acid (PLA), linear and/or branched PEI with differing molecular weights (e.g., 2, 22 and 25 kDa), dendrimers such as PAMAM and polymethoacrylates; lipids including but not limited to cationic liposomes, cationic emulsions, 1,2-dioleoyl-3-trimethylammonium-propane (DOTAP), DOTMA, DMRIE, DOSPA, distearoylphosphatidylcholine (DSPC), DOPE, or DC-chol; peptide based vectors including but not limited to Poly-L-lysine or protamine; or poly(.beta.-amino ester), chitosan, PEI-polyethylene glycol, PEI-mannose-dextrose, DOTAP-cholesterol or PEI-PEG, PEI-PEG-mannose, dextran-PEI, OVA conjugate, PLGA microparticles, or PLGA microparticles coated with PAMAM.

In one embodiment, the delivery vehicle is a glycopolymer-based delivery vehicle, poly(glycoamidoamine)s (PGAAs), that have the ability to complex with various polynucleotide types and form microparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yields exceptional delivery efficiency.

In one embodiment, the delivery vehicle comprises a cationic lipid, e.g., DOTMA, 2,3-dioleyloxy-N-[2-spermine carboxamide] ethyl-N,N-dimethyl-1-propanammonium trifluoracetate (DOSPA, Lipofectamine); DOTAP; N-[1-(2,3-dimyristloxy) propyl]; N,N-dimethyl-N-(2-hydroxyethyl) ammonium bromide (DMRIE), DC-Chol; dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) may enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation efficiency although complex formation with PLL may also increase the encapsulation efficiency. Other cationic materials, for example, PEI, DOTMA, DC-Chol, or CTAB, may be used to make microparticles.

In one embodiment, no delivery vehicle is employed, e.g., nutrient material is employed alone or with a scaffold.

Exemplary properties of a scaffold for use in delivering the nutrient supplementation/waste scavenging materials to the site of a bone defect, injury or surgical site include at least one of biocompatibility, biodegradability, suitable mechanical properties, and scaffold architecture. Biocompatible scaffold or tissue engineered construct does not elicit an immune response or elicits a negligible immune reaction. A biodegradable scaffold allows for regeneration of tissue at the site of the implant. The scaffold has mechanical properties consistent with the anatomical site into which it is to be implanted. For example, bone or cartilage scaffold must have sufficient mechanical integrity to function from the time of implantation to the completion of the remodeling process. Scaffolds may have an interconnected pore structure and/or high porosity.

Three individual groups of biomaterials, i.e., ceramics, synthetic polymers, and natural polymers, are commonly used in the fabrication of scaffolds. Although not generally used for soft tissue regeneration, there has been widespread use of ceramic scaffolds, such as hydroxyapatite (HA) and tricalcium phosphate (TCP), for bone regeneration applications. Ceramic scaffolds are typically characterized by high mechanical stiffness, very low elasticity, and a hard, brittle surface. From a bone perspective, they exhibit excellent biocompatibility due to their chemical and structural similarity to the mineral phase of the native bone. The interactions of osteogenic cells with ceramics are important for bone regeneration as ceramics are known to enhance osteoblast differentiation and proliferation.

As described above, numerous synthetic polymers have been used including polystyrene, poly-l-lactic acid (PLLA), polyglycolic acid (PGA) and poly-dl-lactic-co-glycolic acid (PLGA).

Another delivery vehicle embodiment involves the use of biological materials as scaffold biomaterials. Biological materials such as collagen, various proteoglycans, alginate-based substrates, and chitosan have all been used in the production of scaffolds for tissue engineering. Unlike synthetic polymer-based scaffolds, natural polymers are biologically active and typically promote excellent cell adhesion and growth. Furthermore, the natural polymers are also biodegradable and so allow host cells, over time, to produce their own extracellular matrix.

Collagen and collagen-GAG (CG) scaffolds may be altered through physical and chemical cross-linking. Collagen-hydroxyapatite (CHA) scaffolds, collagen-hydroxyapitite (CHA) scaffolds may be useful for bone defects. Suitable biocompatible materials for the polymers include but are not limited to polyacetic or polyglycolic acid and derivatives thereof, polyorthoesters, polyesters, polyurethanes, polyamino acids such as polylysine, lactic/glycolic acid copolymers, polyanhydrides and ion exchange resins such as sulfonated polytetrafluorethylene, polydimethyl siloxanes (silicone rubber) or combinations thereof.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the biocompatible material for the distinct polymer is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm-blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxy phenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly [(dichlorolphosphazenes] or poly [(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer employed as a scaffold may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

In certain embodiments, the nutritional support material employs a slow-elution carrier for controlling the release of the nutrient cocktail, such that the release half-life is between about 30 minutes and 14 days, inclusive of endpoints. In certain embodiments, the release half-life is between 1 hour and 7 days. In other embodiments, the release half-life is between 1 hour and 24 hours. In some embodiments, the release half-life is between 1 day and 14 days. In some embodiments, the release half-life is between 30 minutes and hours. In some embodiments, the release half-life is about 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In other embodiments, the release half-life is about 1 day, 2 days, 3 days, 4 days, 5 days, 6 days, 7 days, 8 days, 9 days, 10 days, 11 days, 12 days, 13 days, or 14 days.

Slow-elution carriers are usually made of a membrane or matrix. Matrix type formulations are prepared from either swellable hydrophilic polymers or non-swellable lipophilic excipients, like waxes and lipids. Polymers for use in slow-elution systems are known in the art and include those described herein, including Poly-lactic-co-glycolic acid (PLGA), poly (lactic acid) (PLA), and poly(glycol acid) (PGA). See, du Toit L C, Choonara Y E, Kumar P, Pillay V. Polymeric networks for controlled release of drugs: a patent review. Expert Opin Ther Pat. 2016 June; 26(6):703-17. Epub 2016 Apr. 27, which is incorporated herein by reference. To date, poly-lactic-co-glycolic-acid (PLGA) is the most well-known and widely applied polymer in controlled release systems. This synthetic polymer has found great success due to its biocompatibility, biodegradability, and favorable release kinetics, but also faces stability concerns for protein delivery. Poly-(glycolic acid) (PGA) and poly-(lactic acid) (PLA) along with the copolymer poly-(lactic-co-glycolic acid) (PLGA) are biodegradable synthetic polymers that were discovered as surgical sutures in the 1960's. The successful development of these polymers as surgical sutures led to the expansion of their use as polymeric biomaterials. Since then, the copolymer has been established as the most successful and widely researched polymer for application in controlled release systems and is considered the “gold standard” of biodegradable polymers for controlled delivery systems. PLGA has been used to release a wide range of small molecule drugs, peptides, and proteins, including fertility regulating hormones, growth hormones, steroid hormones, anti-inflammatory drugs, cytokines, chemotherapeutics, antibiotics, narcotic antagonists, insulin, and vaccines. In comparison to other polymers that have been investigated for controlled release, PLGA is relatively easy to process into different device morphologies such as injectable micro-/nanospheres. See, Hines, Daniel J, and David L Kaplan. “Poly(lactic-co-glycolic) acid-controlled-release systems: experimental and modeling insights.” Critical reviews in therapeutic drug carrier systems vol. 30,3 (2013): 257-76, which is incorporated herein by reference.

As used herein, the term “microparticles” refers to particles of about 0.1 μm to about 5 mm in size, about 1 μm to about 100 μm, about 0.5 μm to about 50 μm, 0.5 μm to about 20 μm in size, particles of about 1 μm to about 10 μm in size, about 5 μm in size, or mixtures thereof. All ranges include endpoints and all integers therebetween.

As used herein, the term “nanoparticles” refers to particles of about 0.1 nm to about 1 μm, 1 nm to about 1 μm, about 10 nm to about 1 μm, about 50 nm to about 1 μm, about 100 nm to about 1 μm, about 250-900 nm in size, or, advantageously, about 600-800 nm. All ranges include endpoints and all integers therebetween.

The commonly used materials in lipid carriers, prepared by various melt techniques, are beeswax and carnauba wax. These waxes contain a wide group of chemicals such as glycerides, fatty acids, fatty alcohols and their esters. These are widely used as release retardants (coatings) in the design of sustained release beads, tablets, suspensions, implants, and microcapsules. The advantages of waxes include good stability at various pH and moisture levels, well-established safe application in humans due to their non-swellable and water insoluble nature, minimal effect on food in the gastrointestinal tract, and no dose dumping. In one embodiment, the carrier is at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or up to 100% a carnauba wax or beeswax. See, Shah, Hossein & Ganji, Fariba & Vasheghani-Farahani, Ebrahim & Shojaosadati, Seyed. (2009). Sustained release of KCl from Beeswax/Carnauba wax microparticles, which is incorporated herein by reference.

Employing inexpensive yet safe biomaterials to embed waste scavenging materials and release nutrient supplementation in a controlled manner addresses the high cost and safety concerns that exist with other approaches. Thus the in vivo approach of applying nutrient supplementation and waste scavenging directly to the site of surgical repair, bone injury, wound etc will further reduce the treatment cost significantly. Still other delivery vehicles are described in US patent application publications, such as No. 2009/0155216, US2019/0144856 and U.S. Pat. Nos. 6,811,776 and 9,180,000, and other references disclosed herein.

In the embodiments described below, one embodiment of a delivery vehicle is poly(lactic-co-glycolic acid) (PLGA) polymer, which is the most widely used encapsulating agent for production of drug or factor-eluting biomaterials due to its favorable biocompatibility, biodegradability, and its relative hydrophobicity that improves in vivo longevity. Furthermore, it is approved for human use by the United States Food and Drug Administration and the European Medicines Agency. PLGA is therefore projected as a favorable scaffold material for the provision of nutritional elusion according to the methods described herein. In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make microparticles.

Other biocompatible materials include synthetic polymers in the form of hydrogels or other porous materials, e.g., permeable configurations or morphologies, such as polyvinyl alcohol, polyvinylpyrrolidone and polyacrylamide, polyethylene oxide, poly(2-hydroxyethyl methacrylate); natural polymers such as gums and starches; synthetic elastomers such as silicone rubber, polyurethane rubber; and natural rubbers, and include poly[.alpha.(4-aminobutyl)]-1-glycolic acid, polyethylene oxide (Roy et al., 2003), polyorthoesters (Heller et al., 2002), silk-elastin-like polymers (Megeld et al., 2002), alginate (Wee et al., 1998), EVAc (poly(ethylene-co-vinyl acetate), microspheres such as poly (D, L-lactide-co-glycolide) copolymer and poly (L-lactide), poly(N-isopropylacrylamide)-b-poly(D,L-lactide), a soy matrix such as one cross-linked with glyoxal and reinforced with a bioactive filler, e.g., hydroxylapatite, poly(epsilon-caprolactone)-poly(ethylene glycol) copolymers, poly(acryloyl hydroxyethyl) starch, polylysine-polyethylene glycol, an agarose hydrogel, or a lipid microtubule-hydrogel.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), poly(D,L-lactide-coglycolide), poly(lactic acid colysine) and polycaprolactone.

The compositions of the invention may also be for administration in biocompatible organic or inorganic matrices including, but not limited to, collagen or fibrinogen matrices. It is envisaged that such matrices may act as carriers of the composition in an appropriate formulation or may aid in the promotion of tissue healing by augmenting the effects of the composition.

The delivery vehicle may also be a bone repair device or implant coated with the nutrient materials (e.g., as microparticles within a carrier) and/or waste scavenging materials. Suitable devices and implants are described, e.g, in US patent application publication Nos. 2019/0209327 and US 2019/0021862, among others and incorporated by reference herein.

Once the nutritional supplements/waste scavenging materials are prepared with the suitable delivery vehicle, e.g., encapsulated in microparticles, the application of the compositions to the site of bone defect, repair, injury or surgery, can include any suitable means, such as direct surgical implantation, injection, or supplementation of other commonly used surgical implants, scaffolds, or injections to improve both graft and host tissue viability, coating of implants, coating or bathing or spraying of tissues and bone in the surgical site. In one embodiment, physical methods including but not limited to electroporation, sonoporation, magnetoporation, ultrasound or needle injection may be employed to introduce the nutrient supplementation materials and/or waste delivery materials and a delivery vehicle or the materials encapsulated in particles, or a scaffold having complexes of the materials and a delivery vehicle into a selected site of bone defect, injury or repair.

“Encapsulation efficiency” (EE) is defined as the fraction of the substance of interest that is successfully captured within microparticles during fabrication. As an example, expected EE for PLGA microparticles can be variable and depend highly on the fabrication method and type of encapsulant, but generally fall in the region of 30-90% according to prior literature employing PLGA microspheres. See, e.g., Han F Y, Thurecht K J, Whittaker A K, et al. 2016. Bioerodable PLGA-Based Microparticles for Producing Sustained-Release Drug Formulations and Strategies for Improving Drug Loading. Frontiers in Pharmacol., 7:185, incorporated by reference herein. Depending on the delivery vehicle, percentage of active ingredient contained in the formulation may be higher, such as in the case of using the composition alone, or lower, such as in coatings where the bulk of the native implant has no nutritional or waste scavenging function in itself.

In any case, the formulation and delivery vehicle in combination shall ensure sufficient nutrient delivery and waste scavenging capability in a reasonable volume to sustain tissue viability and healing, in a single administration of the composition, for a period of at least 1 hour and up to 4 weeks. Sustained desired effect beyond 4 weeks can be achieved by repeat provisions of the same or another composition, with the total duration of treatment to be determined by a person skilled in the art of treatment.

D. Site of Tissue Defect, Surgery, Repair, Wound or Disease

The “site” in need of the promotion of tissue healing may be any number of areas comprising tissue that is injured, damaged, eroded, brittle, or defective in some other way such that it would benefit from the promotion of cellular stimulation and growth at that site. The promotion of cellular stimulation and growth is envisaged to lead to the acceleration of tissue healing at that site in comparison with the rate of tissue healing seen in patients who are not subject to the present invention. Thus, the site may be a site of bone or other tissue injury. Alternatively, the site may be a site of surgical intervention. The term “site of an injury” includes the site of a fracture of a bone or trauma to soft tissue or organs. By “site of surgical intervention” means that the site may be a site of a surgical repair, involving the making of a surgical incision or re-exploration of an existing incision or open wound, and involving such further intervention as the insertion of an implant into a bone. The site may also be a combination of both a site of injury and a site of surgical invention. In other words, when the site is one of both an injury and a surgical intervention, this may be, for example, the placing of an implant at the site of a fracture. Another embodiment is a site of a percutaneous intervention, which does not necessarily involve the making of a surgical incision or re-exploration of an existing incision or open wound, but rather relies on use of needles and other minimally-invasive means to inject and otherwise manipulate tissue towards the same end of improving tissue healing. Alternative embodiments of such sites that fall within the intended scope of the present invention will be immediately apparent to a person of skill in the art.

The site may be a site requiring bone fusion or comprising damaged bone, eroded bone, or bone defects. Such embodiments may also be found in combination with each other or with a site of injury or surgical intervention. A site where there is damaged and/or eroded bone may be more prone to injury, such as fragility fractures experienced by sufferers of conditions such as osteoporosis. Further, in patients where a site requires bone fusion, one may expect that that site may also be a site of injury, which injury may have led to the requirement for the fusion of a bone. For example, a spinal injury may call for the fusion of two vertebrae to stabilize the spine. Alternatively, it may be a site of other pathology, for example due to degeneration or deformity between vertebrae resulting in the site being treated by surgical fusion of the vertebrae.

By the term “site comprising tissue defects” refers to tissue at that site having a defective composition or structure in comparison to healthy tissue. Such defects may be congenital or they may be acquired through injury, surgery or disease or other cases as would be well known to a person skilled in the art. A site having “tissue defects” or damaged tissue may be assessed, for example, radiographically (e.g. by X-ray or by computational tomography scan), or directly by surgical exposure and visualization, as would be appreciated by a person skilled in the art. Thus, in a preferred embodiment of the present invention the site may be an anatomic space between adjacent spinal vertebrae requiring bone graft or implants to fill a bone defect to effect spinal fusion. The present invention is considered to be particularly useful in spinal fusion where significant periosteal stripping and extensive use of electrocautery routinely creates sites of poor biology.

The present invention is also considered to be useful in repairing less severely damaged tissue, thus allowing the tissue layers that were present at the site before the injury or surgical intervention occurred to be replenished. Such an embodiment is considered to be particularly useful after the insertion of an implant into the bone, where new bone formation is considered to enable the implant to adhere more securely than it would in the absence of the effects of the present invention. An example includes bony ingrowth into the designed ingrowth surface found on many joint replacement implants.

The present invention may allow modulation of bone healing to accelerate as well as improve the quality of healing. This would allow for faster union and improved consolidation of the fracture or implant fixation. In the clinical scenario, there is a race between fracture union/consolidation and implant failure, especially in compromised bone as exemplified by fragility, pathological, or atypical fractures or fracture nonunion. The invention is considered to promote union and consolidation, thereby reducing complications at the fracture or implant site and allow more rapid mobilization of the patient.

In an embodiment of any aspect of the invention the surgical intervention may be an osteotomy. By “osteotomy” we include any surgical procedure where bone is purposefully cut to change its length, alignment, rotation, or position. The present invention is envisaged to provide a means by which the healing process, after such a procedure, may be accelerated.

In certain surgical procedures, bone grafts are utilized to accelerate growth and healing of bone. With the application of the present invention in addition to a bone graft, it is envisaged that the bone healing process may be accelerated further. An example of when a bone graft may be used includes instances when the fusion of bone is required. It is considered that the present invention will not only aid in the acceleration of the healing of a site of grafted bone but also in healing the site where the donor bone has been excised. Thus, in an embodiment the surgical intervention may be the removal of bone from a donor site for a bone graft. In a further embodiment the site of surgical intervention may be the site of a bone graft itself. It is considered that the promotion of bone formation will aid in repairing bone and/or accelerating bone formation at the site of fracture. Promoting fixation may also be useful in osteointegrated implants, e.g., teeth, digits, facial prosthesis, and hearing devices.

E. General Components

“Patient” or “subject” or “individual” as used herein means a mammalian animal, including a human, a veterinary or farm animal, a domestic animal or pet, and animals normally used for clinical research. In one embodiment, the subject of these methods and compositions is a human. In one embodiment, the subject has a tissue injury, tissue repair or tissue disease.

A “pharmaceutically acceptable excipient or carrier” refers to, without limitation, a diluent, adjuvant, excipient, auxiliary agent, or vehicle with which an active agent of the present invention is administered. Pharmaceutically acceptable carriers are those approved by a regulatory agency of the Federal or a state government or listed in the U.S. Pharmacopeia or other generally recognized pharmacopeia for use in animals, and more particularly in humans, can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “treatment” refers to any method used that imparts a benefit to the subject, i.e., which can alleviate, delay onset, reduce severity or incidence, or yield prophylaxis of one or more symptoms or aspects of a tissue injury, tissue repair, or tissue disease as previously described herein. For the purposes of the present invention, treatment can be administered before, during, and/or after surgical treatment. In certain embodiments, treatment occurs after the subject has received surgery. In some embodiments, the term “treating” includes abrogating, substantially preventing the appearance of clinical or aesthetic symptoms of a condition or decreasing the severity and/or frequency one or more symptoms resulting from the tissue injury, surgery, repair or disease.

An “effective amount” is meant the amount of the nutrient supplement materials and/or waste scavenging materials or any optional compositions, sufficient to provide a therapeutic benefit or therapeutic effect after a suitable course of administration. A “therapeutically effective amount” of a compound or a pharmaceutical composition refers to an amount effective to quantifiably prevent, inhibit, treat, or lessen the symptoms of a particular disorder or disease. For example, “therapeutically effective amount” may refer to an amount of a nutrient supplement material sufficient to stimulate cell or tissue repair at the site of surgery in a treated subject faster or more efficiently than a similar subject receiving no treatment during surgery. It should be understood that the “effective amount” for the nutrient materials vary depending upon the materials selected for use in the method, as well as the severity and extent of tissue injury, wound, or disease. It should be understood that the “effective amount” for the waste scavenging materials vary depending upon the materials selected for use in the method. Regarding doses, it should be understood that essential nutrient materials by their definition are safe over a large range of dosages, as known in the art for individual nutrients. As the present invention does not concern the systemic organism, but rather only local or regional tissue damage, dosages are not generally determined by body weight. With an injectable a physician or nurse can inject a calculated amount by filling a syringe from a vial with this amount. In contrast, implants or scaffolds may contain fixed dosage forms. Some dose ranging studies with small molecules use mg/kg, but other dosages can be used by one skilled in the art, based on the teachings of this specification. In one embodiment an effective amount for the nutrient composition delivered as a microparticle or implant includes without limitation about 0.001 to about 500 mg/kg subject body weight. In another embodiment, the subject may be injected with 100 microgram to 50 grams in a delivery vehicle during surgery and after surgery is completed, local injections may be repeated in volumes of 1-20 milliliters or 5-10 cc.

The terms “a” or “an” refers to one or more. For example, “an amino acid” is understood to represent one or more such amino acids. As such, the terms “a” (or “an”), “one or more,” and “at least one” are used interchangeably herein.

As used herein, the term “about” means a variability of plus or minus 10% from the reference given, unless otherwise specified.

The words “comprise”, “comprises”, and “comprising” are to be interpreted inclusively rather than exclusively, i. e. , to include other unspecified components or process steps. The words “consist”, “consisting”, and its variants, are to be interpreted exclusively, rather than inclusively, i.e., to exclude components or steps not specifically recited.

Technical and scientific terms used herein have the same meaning as commonly understood by one of ordinary skill in the art to which this invention belongs and by reference to published texts, which provide one skilled in the art with a general guide to many of the terms used in the present application. The definitions contained in this specification are provided for clarity in describing the components and compositions herein and are not intended to limit the claimed invention.

### II. Compositions

Thus, in one embodiment, a composition for increasing healing at the site of a tissue injury or wound comprises an effective amount of a nutritional support material, and an optional effective amount of a waste scavenging material in a physiologically acceptable delivery system suitable for delivery to the site of the tissue defect, tissue injury, tissue disease, or surgical site. The compositions may be formulated as appropriate for the type of injury or surgical intervention in question. Appropriate formulations will be evident to a person of skill in the art and may include, but are not limited to, the group comprising a solid for implant, a liquid for injection, a powder, a chip, a pellet, a microparticle, a nanoparticle, a capsule, a cream, a gel, a lotion, a paste, a coating, an impregnation of an existing implant or tissue, a combination thereof. The formulations may conveniently be presented in unit dosage form and may be prepared by any of the methods well known in the art of pharmacy. Such methods include the step of bringing into association the active ingredient (formulation of the invention) with a suitable delivery vehicle as discussed above. In general, the formulations are prepared by uniformly and intimately bringing into association the active nutrient ingredients (and other waste scavenging ingredients) with liquid carriers or finely divided solid carriers or both, and then, if necessary, shaping the product.

The formulation may comprise a controlled release preparation which is biocompatible, liquid at low temperature, so suitable for injection, but assumes gel characteristics at body temperature. By “low temperature” we include the meaning of a temperature lower than typical, healthy, body temperature. Examples of such formulations include those based on Pluronic gels (F1 27) and ReGel™, as will be well known to those skilled in the art. The Pluronic F127 could be combined with cross-linked polyethylene glycol fibrinogen conjugates and the nutrient materials/waste scavenging materials combined with these matrices would be optimally delivered to the site of desired activity.

The compositions of the invention when introduced into the suitable delivery vehicle are for administering locally at the site where tissue healing or formation is required. They may be used in the form of a sterile aqueous solution which may contain other substances, for example, enough salts or glucose to make the solution isotonic with blood. The aqueous solutions should be suitably buffered (preferably to a pH of from 3 to 9), if necessary. In one embodiment, the pH of the formulation is about 7.4 for application to the site of tissue injury/surgical site. The preparation of suitable formulations under sterile conditions is readily accomplished by standard pharmaceutical techniques well-known to those skilled in the art.

Formulations suitable for local administration include aqueous and non-aqueous sterile injection solutions which may contain anti-oxidants, buffers, antibiotics, antifungals, antiparasitics, and other solutes which render the formulation isotonic with the intended recipient tissue, immunologically acceptable, and prophylactic against infection; and aqueous and non-aqueous sterile suspensions which may include suspending agents and thickening agents. The formulations may be presented in unit-dose or multi-dose microparticle aliquots, and may be stored in a freeze-dried (lyophilized) condition requiring only the addition of the sterile liquid carrier, for example normal saline for injections or implantation, immediately prior to use. Extemporaneous injection solutions and suspensions may be prepared from sterile powders, granules and tablets of the kind previously described. See, e.g., US2014/0056960. These formulations are then prepared as microparticles, gels or in other delivery systems as disclosed herein.

In still another embodiment, a composition for implantation into the site of surgery is coated, admixed, or filled with a nutritional support material or a waste scavenging material for gradual release after implantation into the site of tissue defect or injury.

### III. Methods

The invention provides a method of increasing healing of tissue injury or wound comprises introducing nutritional support material at the site of tissue repair, site of tissue surgery, or wound to improve cell survival in the wound environment. In one embodiment, the material is introduced during a surgical procedure prior to closure of said site of tissue repair or wound. In another embodiment, the material is introduced to the site of tissue repair or wound either prior to or without requiring a surgical procedure. In still another embodiment, the material is introduced to the site of tissue repair or wound subsequent to a surgical procedure after closure of said site of tissue repair or wound.

As disclosed above, the “material” can include any nutritional support material described herein, including one or more of sugars, amino acids, vitamins, fatty acids, minerals, salts, and nucleic acids and/or a cell culture medium. The material should maintain a physiological pH. In another embodiment, the method further comprises introducing waste scavenging materials at the site of tissue repair or wound to sequester toxins that could negatively affect tissue healing or cell survival. The waste scavenging material is as described above and can include, in one embodiment, activated charcoal. In another embodiment, activated charcoal is coated in cellulose. In another embodiment, one or more flavonoid compounds is included as a waste scavenging material.

The nutritional support material is in one embodiment, contained within a delivery composition, optionally with the waste scavenging material. In an alternative embodiment, the nutrient support material is contained within one formulation or delivery vehicle and the waste scavenging material is contained in a separate delivery vehicle. Such delivery vehicles may be the same or different for the two types of compositions for delivery to tissue. Such delivery vehicles are as described above and include a solid for implant, a liquid for injection, a powder, a chip, a pellet, a microparticle, a nanoparticle, a capsule, a cream, a gel, a lotion, a paste, a coating, an impregnation of an existing implant or tissue, a combination thereof, or any biologically acceptable delivery apparatus. In one specific embodiment, the delivery vehicle is a microparticle comprised of a polymer such as PLGA. Still other vehicles noted above may be used.

According to one embodiment, the method involves bathing, coating, layering, injecting, or spraying the site of the tissue injury or wound with an effective amount of the nutritional support material and applying the optional waste scavenging material in the same manner The site of such application may be the surgical field including the site of the tissue defect and associated tissue types including but not limited to bone, periosteum, muscle, tendon, ligaments, cartilage, fascia, vasculature, granulation tissue, fat, and dermis. In another embodiment, the compositions are introduced by inserting at the site of the bone injury or wound prior to surgical closure a delivery vehicle such as those described herein. In one embodiment, the delivery vehicle is capable of delivering in vivo an effective amount of said nutrient support material and optional waste scavenging material at the site of the injury or repair or regrowth.

In still another embodiment, the method involves injecting into the site of the tissue injury or wound a nutrient or waste scavenging composition that is capable of delivering in vivo an effective amount of said support material and optional waste scavenging material. The composition can include a liquid, gel, suspension, or matrix deliverable in this manner In another embodiment, injection of the compositions may occur post-surgical closure at times and doses selected by the surgeon and physician. In one embodiment, the injecting occurs after surgical closure of the site of bone injury or wound.

The method may be applied before, during, with or after any suitable surgical procedure, such as bone grafting, tissue repair, spinal fusion, or insertion of an implant into a bone defect, among others identified herein.

In the case of local administration circumstance, administration is into any tissue cavity within which the formulation may improve tissue healing. This can be supplemented by additional interventions of the same. That is to say, the step may be made to administer an implant or other delivery vehicle locally to the region requiring tissue viability support during surgery, and then following up post-operatively with directed injection or administration to the site of injury or surgical repair. In still other embodiments, the nutrient and/or waste scavenging materials may be admixed and delivered with bone graft materials.

Other embodiments of the compositions and methods are believed to be within the skill of the art based upon the teachings provided herein.

### IV. EXAMPLES

The following examples disclose specific embodiments of the methods and compositions above described and should be construed to encompass any and all variations that become evident as a result of the teaching provided herein. In these examples, Dulbecco's Modified Eagle Medium (DMEM) is an enriched cell culture medium. PLGA together with DMEM provide a reliable example of ingredients for the production of nutrient microparticles.

### Example 1

### Development of Nutrient-Eluting and Waste-Scavenging Microparticles for Sustaining Cellular Viability

As described below, we have developed a process for producing nutrient-eluting microparticles. Microparticle characterization, including the measurement of Encapsulation Efficiency (EE), elution profile, and microscopic analysis are performed as part of baseline characterization. Charcoal-based waste-scavenging microparticles may optionally be used synergistically with nutrient provision to improve cell longevity.

A. Preparation of PLGA-loaded Nutrient Microparticles: A solid-in-oil-in-water method is used. In brief, the primary emulsion uniformly suspends nutrient solids in a bath of dissolved polymer. Following direct addition of the primary emulsion to a surfactant (for example, PVA; polyvinyl alcohol), spherical droplets of roughly uniform size and shape result from the hydrophilic-hydrophobic interaction of the primary emulsion with the water bath in presence of surfactant. This final mixture, known as the secondary emulsion, permits not only formation of the microparticles but also triggers particle hardening through gradual extraction of the organic solvent from the droplets. Eventually, PLGA particles remain in floating surfactant solution and can be removed by gravity separation.

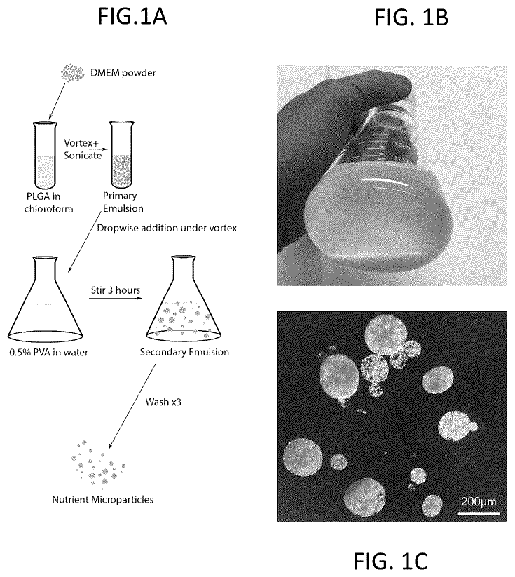

FIGS. 1A-1C demonstrates an example of the stepwise fabrication protocol with darkfield microscopic analysis. Chloroform is used for the primary emulsion and 0.5% PVA is used as the surfactant for the secondary emulsion. Under a chemical fume hood at room temperature, fine DMEM powder is suspended in 10× w/v chloroform containing an equal weight of dissolved PLGA followed by a 10-second bath sonication. This forms the solid-in-oil primary emulsion.

Under medium speed vortex, the primary emulsion is added dropwise to a test tube containing an excess of 1% w/v polyvinyl alcohol in deionized water. This solid-in-oil-in-water secondary emulsion is then stirred gently at 200 rpm in a magnetic stirrer for 3 hours until microparticles have sufficiently hardened. Excess supernatant is decanted, and the remaining microparticles are transferred to a small glass scintillation vial and washed three times with ice-cold deionized water, allowing particles to settle in between. Final wash is discarded following a 5-minute centrifugation at 1000 g. The microparticles are stored at -until lyophilization to create the final microparticles.

B. Preparation of charcoal microparticles: Waste scavenging microparticles are produced using cellulose-coated activated charcoal. Following a published protocol of Park T J, et al. 2008. Heparin-cellulose-charcoal composites for drug detoxification prepared using room temperature ionic liquids. Chem. Commun.:5022-5024, cellulose-coated charcoal microparticles are produced using a solid-in-ionic liquid-in-water method employing 1-Butyl-3-methylimidazolium chloride ([BmIm][Cl]) as the solvent in the primary emulsification. Cellulose is added to [BmIm][Cl] and heated to 70° C. for 30 min to fully dissolve. Uncoated activated charcoal beads of 50-150 μm size are stirred into this solution and this primary emulsification is added dropwise to an ethanol bath and stirred for 24 hours. The beads are washed with double distilled water for 3 hours and dried in a dessicator and stored in a moisture-free fashion.

C. Nutrient microparticle characterization: Microparticles of 50 to 300 microns in diameter are reliably produced using the protocol described. Darkfield microscopy is used to size microparticles immediately following fabrication to ensure consistency in production (FIGS. 1A-1C).

D. Encapsulation Efficiency (EE): EE is measured by chemical extraction of a pre-weighed amount of nutrient microparticle by dissolution of the particles in chloroform followed by vigorous extraction with deionized water. Measurement of DMEM content is performed by absorbance spectrophotometry using phenol red—a component of DMEM media—as an indicator of DMEM content. Measurement of peak absorbance is performed at 560 nm after alkanization using concentrated sodium hydroxide.²³ A serial dilution of known concentration of DMEM in water is used as the standard, and EE is calculated from this reference.

### Example 2

### Microparticles Sustain Cell Viability and Maintain Their Regenerative Phenotype In Vitro

A viability assay is needed to demonstrate that these microparticles sufficiently support cell viability and can maintain stem cell multipotency. Using a human mesenchymal stem cell line, one can demonstrate sufficiency of the nutrient-eluting microparticles, or the waste-scavenging microparticles (or both) in sustaining cell viability in vitro. To demonstrate maintenance of the regenerative phenotype, one can evaluate the surviving cells' ability to differentiate into osteogenic, chondrogenic, and adipogenic pathways over time.

Towards this end, co-culturing experiments employing a human mesenchymal stem cell line are used to demonstrate the ability of the microparticles to maintain and sustain cell viability up to 21 days. Cells then undergo multipotency assessment at time point intervals to demonstrate persistence of multipotency. Success of the coculturing assay indicates sufficiency of the nutrient and/or waste scavenging microparticles in maintaining their regenerative phenotype and cell vigor.

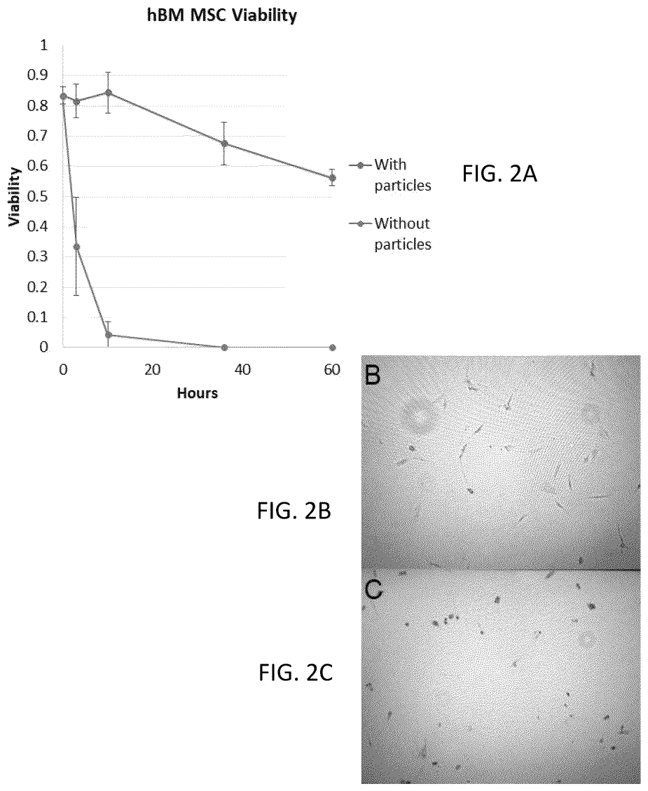

The preliminary cell viability assay was performed using a primary human bone marrow mesenchymal stem cell (hBM MSC) culture (FIGS. 2A-2C). Viability assays are performed to establish the efficacy of the microparticles in promoting cell viability in more than one cell type. The hMSC cell line has the added benefit of being able to harvest cells at various time intervals to assess the maintenance of their multipotent phenotype. Cells were placed in either 1×HEPES-buffered saline (HBSS) alone or 1×HBSS+microparticles at a ratio of 0.65 mg microparticles per 1000 cells. Cultures were stained with Trypan Blue solution for viability calculation over multiple time points up to 60 hours. Drastic difference in cell viability was encountered between the two conditions. This assay demonstrates the necessity of nutrition for the viability of hBM MSCs.

### Example 3

### Wax-Coated Slow Elution Nutrient Particles

Microparticles were produced using a DMEM-based nutrient composition using a dry-coating method with 95%-100% carnauba wax to achieve solid-core slow-elution nutrient microparticles. Solid DMEM particles were first produced with the addition of a small dose of antibiotic-antifungal mixture of penicillin, streptomycin, and amphotericin to suppress unwanted bacterial growth. This was performed by vigorous mechanical agitation of solid DMEM particles against carnauba wax powder until particles are fully coated, with or without weighted beads as mechanical catalysts.

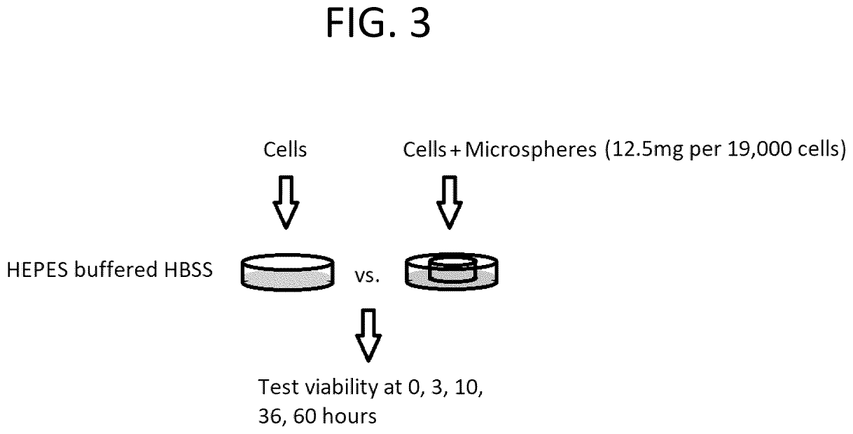

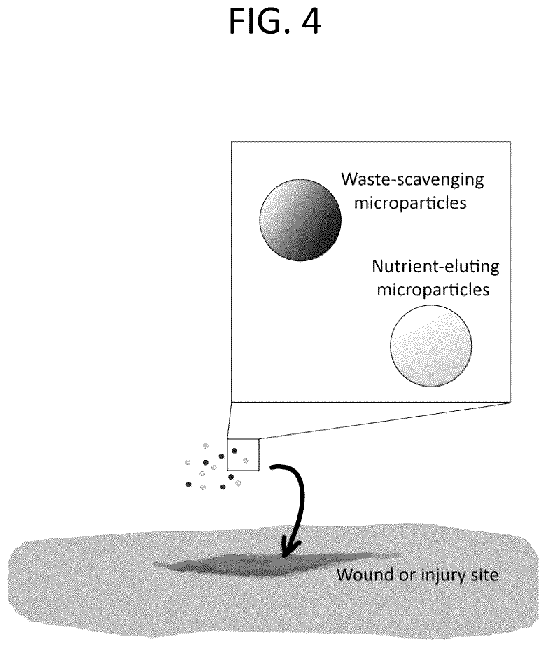

To demonstrate cell viability up to 4 weeks, aliquots of 20,000 cells in combination with nutrition microspheres, biocompatible charcoal particles, or both, are added to separate wells of 12-well culture plates (FIG. 3). Ratio of microparticles to cells are titrated to 0.5, 2, and 5mg per 1000 cells for optimization of microparticle concentration. Culture medium is HEPES-buffered saline with or without fetal bovine serum (ThermoFisher, Waltham, MA) with control wells containing culture medium with blank PLGA particles only. Triplicates are harvested at 0, 8, and 24 hours, and then every few days until day 25 to trend cell viability and proliferation. The co-culturing experiment is performed using tissue culture plates with Transwell inserts (Corning, Corning, NY) in 5% CO₂ in order to provide separation of the microparticles from the cell culture monolayer. This permits ease of monolayer imaging without light interference from the microparticles.

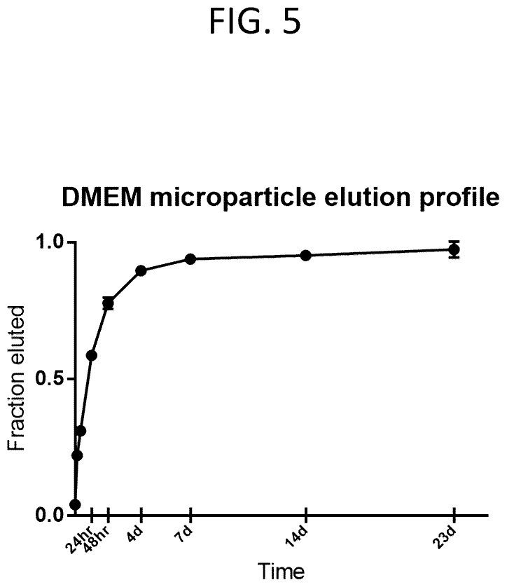

Nutrient elution of microparticles produced using this method demonstrated a logarithmic profile with a half-life of approximately 20 hours and a desirable early burst-release (FIG. 5).

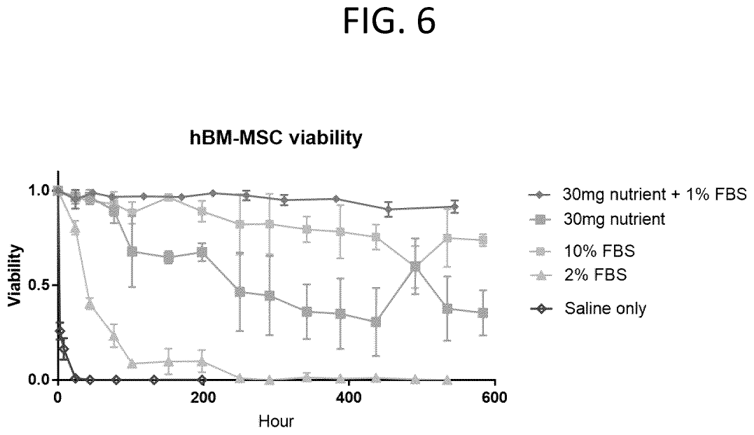

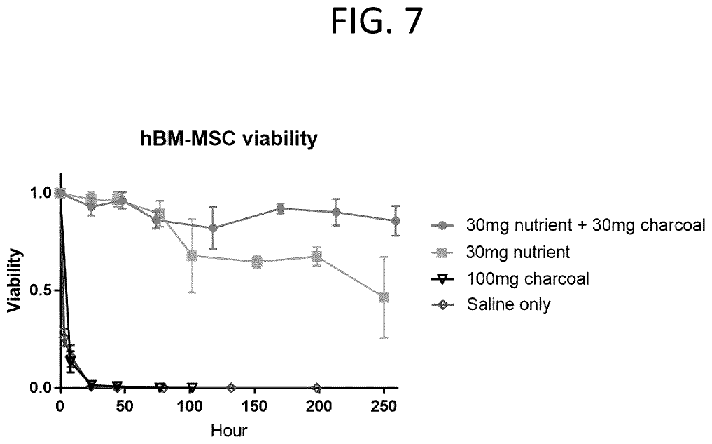

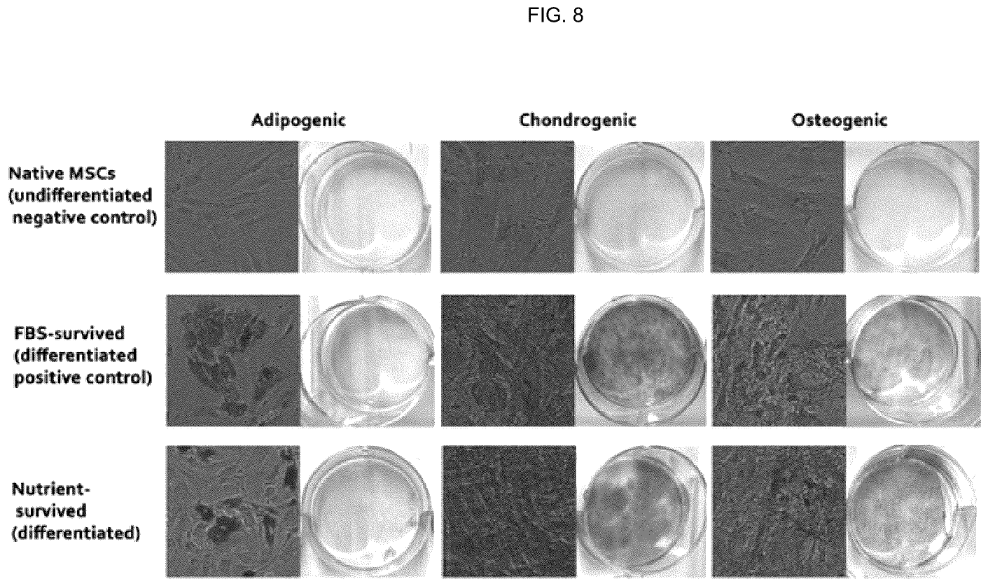

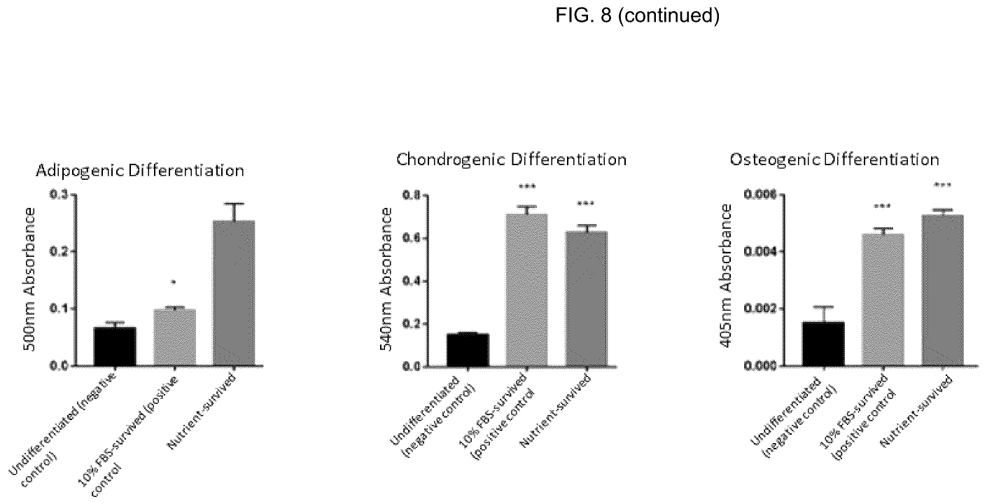

Using these wax-coated microparticles, an in vitro viability study was performed using monolayer human bone marrow-derived mesenchymal stem cells as a model cell type. Cells were placed in co-culture with or without nutrient microparticles in saline. Positive control was provided using 2% and 10% fetal bovine serum (FBS) in saline. Synergistic effect with nutrient microparticles plus 1% serum was tested as well. Cell culture viability testing over 3 weeks demonstrated a strong viability response to nutrient microparticles alone, as well as not just additive, but synergistic effect when used in conjunction with just 1% serum (FIG. 6). Additional synergism was demonstrated when used in conjunction with activated charcoal microparticles, suggesting a role of waste scavenging for improving cell viability (FIG. 7). To ensure that there has been no loss of phenotype, confirmation of pluripotent activity of 3 week-survived cells was performed (FIG. 8). Overall results suggest that nutrient microparticles have good effect in vivo, where they would act in synergy with extracellular fluid (containing similar nutritive components of native serum), to provide cell-sustaining support.

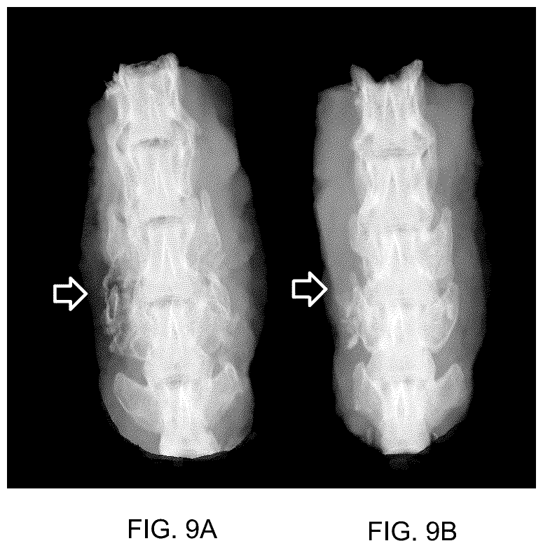

To test in vivo efficacy of these wax-coated nutrient microparticles in sustaining tissue survival, a clinically-simulated animal study was performed. Nutrient microparticles were used to test its ability to sustain bone graft survival in a rat model for lumbar spinal fusion. In this model, iliac bone autograft was harvested from the pelvis of animals undergoing surgery. This bone graft was mixed with either blank microparticles (containing wax only; control group) or an equal amount of wax-coated nutrient microparticles (experimental group). Lumbar spinal fusion was then performed by exposing the transverse processes of lumbar vertebral levels L4 and L5, burring the host bone surfaces, and placing the microparticle-bone graft mix into this host site. Radiographic healing was compared by X-ray analysis. Results of this analysis demonstrated an evident increase in bone production in the nutrient microparticle-supplanted animals as compared to control (FIG. 9).

These example experiments demonstrate the inventors' hypotheses that supplementation of fundamental factors required for maintaining cell homeostasis in a fresh surgical site improves graft viability and thus improves rates of tissue healing. Specifically, prior to the formation of a soft callus after bony disruption, a period of time passes with the graft being isolated from reliable intrinsic blood supply while being bathed in local tissue inflammation.⁷ Thus, supplementation of nutrition and sequestration of cellular waste, such as in the form of locally supplemented microparticles, prolongs cell viability in an otherwise unfavorable host environment.

Each and every patent, patent application, and publication, including websites cited throughout specification are incorporated herein by reference. While the invention has been described with reference to particular embodiments, it will be appreciated that modifications can be made without departing from the spirit of the invention. Such modifications are intended to fall within the scope of the appended claims.

### Specific Embodiments

1. A nutritional support material for supplementing tissue survival comprising
   a) a slow-elution carrier for controlling nutrient release; and
   b) a nutrient cocktail that comprises glucose;
   and wherein the slow-elution carrier has a release half-life of between 30 minutes and 14 days.
2. The nutritional support material according to embodiment 1, wherein the nutritional support material is implantable or injectable.
3. The nutritional support material according to embodiment 1 or embodiment 2, wherein the nutrient cocktail comprises DMEM, EMEM, RPMI, IMDM, Ham's F10, or Ham's F12 media powder.
4. The nutritional support material according to any one of embodiments 1 to 3, wherein the material is in the form of a microparticle between 1 micron and 5 mm in diameter.
5. The nutritional support material according to any one of embodiments 1 to 4, wherein the carrier is a carrier polymeric matrix, optionally PLGA.
6. The nutritional support material according to any one of embodiments 1 to 3, wherein the carrier is a coating.
7. The nutritional support material according to embodiment 6, wherein the coating comprises at least 95% wax or a wax-like substance.
8. The nutritional support material according to any one of embodiments 1 to 7, wherein the material is in the form of impregnation into a porous implant.
9. The nutritional support material according to any one of embodiments 1 to 7, wherein the material is in the form of chips, spheres or blocks between 2 cm and 20 cm in size.
10. The nutritional support material according to any one of embodiments 1 to 7, wherein the material is in the form of nanoparticles between 5 nanometers and 1 micron.
11. The nutritional support material according to any one of embodiments 1 to further comprising an antibiotic and/or antifungal to suppress undesirable contamination growth.
12. A method of supplementing tissue survival relating to injury, disease, or surgery, comprising administering the nutritional support material of any one of embodiments 1 to 11 to the site of tissue injury, disease, surgery, or wound.
13. A method of supplementing autograft bone chips, the method comprising administering the nutritional support material of any one of embodiments 1 to 11 with a bone graft.
13. A method of supplementing cell therapy, the method comprising administering the nutritional support material of any one of embodiments 1 to 11 with a cell therapy.
14. A method of increasing healing of tissue injury, disease, surgery, or wound comprising introducing the nutritional support material of any of embodiments 1 to 11 at the site of tissue injury, disease, surgery, or wound to improve cell survival in the wound environment.
15. The method according to embodiment 14, wherein said material is introduced during a surgical procedure prior to closure of said site of tissue injury, disease, surgery, or wound.
16. The method according to embodiment 14, wherein said material is introduced to the site of tissue injury, disease, surgery, or wound prior to a surgical procedure.
17. The method according to embodiment 14, wherein said material is introduced to the site of tissue injury, disease, surgery, or wound subsequent to a surgical procedure after closure of said site of tissue injury, disease, surgery, or wound.
18. The method according to embodiment 14, wherein said material is introduced to the site of tissue injury or wound without surgical intervention.
19. The method according to any of embodiments 14 to 18, wherein the nutritional support material comprises a mixture of one of more of sugars, amino acids, vitamins, fatty acids, minerals, salts, and nucleic acids and a cell culture medium and has a physiologic pH.
20. The method according to any one of embodiments 14 to 19, wherein said method further comprises introducing waste scavenging materials at the site of tissue injury, disease, surgery, or wound to remove toxins from the site of tissue injury, disease, surgery, or wound.
21. The method according to embodiment 20, wherein the waste scavenging material comprises activated charcoal.
22. The method according to any one of embodiment 14 to 21, wherein said nutritional support material is contained within a delivery composition, optionally with said waste scavenging material.
23. The method according to embodiment 22, wherein said delivery composition is a solid for implant, a liquid for injection, a powder, a chip, a pellet, a microparticle, a nanoparticle, a capsule, a cream, a gel, a lotion, a paste, a coating, an impregnation of an existing implant or tissue, a combination thereof , or any biologically acceptable delivery apparatus.
24. The method according to embodiment 23, wherein said microparticle is comprised of one or more of PLGA or other encapsulating polymer.
25. The method according to any one of embodiment 14 to 24, wherein said introducing step comprises one or more of bathing, coating, layering, injecting, impregnating, or spraying the site of the tissue injury, disease, surgery, or wound with an effective amount of said support material with optional waste scavenging material.
26. The method according to any one of embodiment 14 to 24, wherein said introducing step comprises inserting at the site of the tissue injury, disease, surgery, or wound prior to surgical closure a delivery composition that is capable of delivering in vivo an effective amount of said support material and optional waste scavenging material.
27. The method according to any one of embodiment 14 to 24, wherein said introducing step comprises injecting into the site of the tissue injury, disease, surgery, or wound a delivery composition that is capable of delivering in vivo an effective amount of said support material and optional waste scavenging material.
28. The method according to embodiment 24, wherein said injecting occurs before, during, or after surgical closure of the site of said tissue injury or wound or performed without surgical intervention.
29. The method according to any one of embodiments 14-25, wherein the surgical procedure is bone grafting, tissue repair, spinal fusion, or insertion of an implant into a bone defect.
30. A composition for increasing healing at the site of a tissue injury, disease, surgery, or wound comprising an effective amount of a nutritional support material and an optional effective amount of a waste scavenging material in a physiologically acceptable delivery system.
31. A composition for implantation into the site of tissue injury, disease, surgery, or wound that is coated with a nutritional support material or a waste scavenging material.

### REFERENCES

1. Gould S E, et al. 2000. Cellular contribution of bone graft to fusion. J. Orthoped. Res., 18:920-927.
2. Kruyt M C, et al. 2004. Osteogenicity of autologous bone transplants in the goat. Transplantation 77:504-509.
3. Holton J, et al. 2016. The Basic Science of Bone Marrow Aspirate Concentrate in Chondral Injuries. Orthopedic reviews 8:6659.
4. Hernigou P, et al. 2005. Percutaneous autologous bone marrow grafting for nonunions. Influence of the number and concentration of progenitor cells. J. Bone Joint Surg. Amer., 87:1430-1437.
5. Tuchman A, et al. 2017. Autograft versus Allograft for Cervical Spinal Fusion: A Systematic Review. Global Spine J., 7:59-70.
6. Sen M K, Miclau T. 2007. Autologous iliac crest bone graft: should it still be the gold standard for treating nonunions? Injury 38 Suppl 1:S75-80.
7. Marsell R, Einhorn T A. 2011. The biology of fracture healing. Injury 42:551-555.
8. Han F Y, Thurecht K J, Whittaker A K, et al. 2016. Bioerodable PLGA-Based Microparticles for Producing Sustained-Release Drug Formulations and Strategies for Improving Drug Loading. Frontiers in Pharmacol., 7:185.
9. Allahyari M, Mohit E. 2016. Peptide/protein vaccine delivery system based on PLGA particles. Human vaccines & Immunotherapeutics, 12:806-828.
10. Yao T, Asayama Y. 2017 Animal-cell culture media: History, characteristics, and current issues. Reproduc. Med. and Biol., 16:99-117.
11. Derlet R W, Albertson T E. 1986. Activated charcoal—past, present and future. Western J. Medicine 145:493-496.
12. Sandeman S R, et al. 2017. A haemocompatible and scalable nanoporous adsorbent monolith synthesised using a novel lignin binder route to augment the adsorption of poorly removed uraemic toxins in haemodialysis. Biomed. Materials 12:035001.
13. Pan M, Staden Jv. 1998. The use of Charcoal in in vitro culture—A review. Plant Growth Reg. 26:155-163.
14. Thomas T D. 2008. The role of activated charcoal in plant tissue culture. Biotech. Adv., 26:618-631.
15. Park T, et al. 2011. Biocompatible charcoal composites prepared by ionic liquids for drug detoxification. Macromolecular Research 19:734-738.
16. Winchester J. 1983. Hemoperfusion. In: Drukker W, Parsons F, Maher J editors. Replacement of Renal Function by Dialysis: A textbook of dialysis, 2 ed. Netherlands: Springer
17. Zeerleder S, et al. 2002. Effect of low-molecular weight dextran sulfate on coagulation and platelet function tests. Thrombosis research 105:441-446.
18. Agostino D. 1976. The effect of high molecular dextran on coagulation and metastases. Tumori 62:245-254.
19. Keshavarzi S, et al. 2013. Clinical experience with the surgical family of absorbable hemostats (oxidized regenerated cellulose) in neurosurgical applications: a review. Wounds: a compendium of clinical research and practice 25:160-167
20. McCall R L, Sirianni R W. 2013. PLGA nanoparticles formed by single- or double emulsion with vitamin E-TPGS. J. Visualized Exp. 51015.
21. Mu L, Feng S S. 2003. A novel controlled release formulation for the anticancer drug paclitaxel (Taxol): PLGA nanoparticles containing vitamin E TPGS. J. Controlled Release, 86:33-48.
22. Park T J, et al. 2008. Heparin-cellulose-charcoal composites for drug detoxification prepared using room temperature ionic liquids. Chem. Commun.:5022-5024.
23. Held P. 2018. Using phenol red to assess pH in tissue culture media. BioTek Live Cell Imaging Application Note.
24. Shive M S, Anderson J M. 1997. Biodegradation and biocompatibility of PLA and PLGA microspheres. Adv. Drug Deliv. Rev. 28:5-24.
25. Yano, K. et al, 2015 Fate, origin, and roles of cells within free bone grafts., J. Orthop. Sci, 20:390-396
26. Bibby, S R S and J P G Urban, 2004. Effect of nutrient deprivation on the viability of intervertebral disc cells, Eur Spine J. 13:695-701
27. Panse N, et al. Diet, wound healing and plastic surgery. Indian J Plast Surg. 2013 January; 46(1):161-3.
28. Maroz N, Simman R Wound Healing in Patients With Impaired Kidney Function. J Am Coll Clin Wound Spec. 2013 April; 5(1):2-7.
29. Karalis M, et al. Effect of experimentally induced liver cirrhosis on wound healing of the post-extraction tooth socket in rats. Eur Surg Res. 2008; 40(2):190-6.
30. Cordero-Espinoza L, Huch M. The balancing act of the liver: tissue regeneration versus fibrosis. J Clin Invest. 2018 Jan. 2; 128(1):85-96.
31. Kueper J, et al. Evidence for the adverse effect of starvation on bone quality: a review of the literature. Int J Endocrinol. 2015; 2015:628740.
32. Meesters D M, et al. Malnutrition and Fracture Healing: Are Specific Deficiencies in Amino Acids Important in Nonunion Development? Nutrients. 2018 Oct. 31; 10(11).
33. Medical Advisory S. Management of chronic pressure ulcers: an evidence-based analysis. Ont Health Technol Assess Ser. 2009; 9(3):1-203.
34. Bauer J D, et al. The effectiveness of a specialised oral nutrition supplement on outcomes in patients with chronic wounds: a pragmatic randomised study. J Hum Nutr Diet. 2013 October; 26(5):452-8
35. Brown S A, et al. Oral nutritional supplementation accelerates skin wound healing: a randomized, placebo-controlled, double-arm, crossover study. Plast Reconstr Surg. 2004 July; 114(1):237-44.
36. Stojanovic M D, et al. Enhanced Recovery after Vascular Surgery. Front Med (Lausanne). 2018; 5:2.
37. Jabbour J, et al. Role of nutritional indices in predicting outcomes of vascular surgery. J Vasc Surg. 2019 August; 70(2):569-79 e4
38. Rahm D A guide to perioperative nutrition. Aesthet Surg J. 2004 July-August; 24(4):385-90
39. Singh, M et al. 2000, January Cationic microparticles: A potent delivery system for DNA vaccines. Proc. Natl Acad Sci. USA 97(2):811-816.

1. A nutritional support material for supplementing tissue survival comprising
a) a slow-elution carrier for controlling nutrient release; and
b) a nutrient cocktail that comprises glucose;
and wherein the slow-elution carrier has a release half-life of between 30 minutes and 14 days.

2. The nutritional support material according to claim 1, wherein the nutritional support material is implantable or injectable.

3. The nutritional support material according to claim 1 or claim 2, wherein the nutrient cocktail comprises DMEM, EMEM, RPMI, IMDM, Ham's F10, or Ham's F12 media powder.

4. The nutritional support material according to any one of claims 1 to 3, wherein the material is in the form of a microparticle between 1 micron and 5 mm in diameter.

5. The nutritional support material according to any one of claims 1 to 4, wherein the carrier is a carrier polymeric matrix, optionally PLGA.

6. The nutritional support material according to any one of claims 1 to 3, wherein the carrier is a coating.

7. The nutritional support material according to claim 6, wherein the coating comprises at least 95% wax or a wax-like substance.

8. The nutritional support material according to any one of claims 1 to 7, wherein the material is in the form of impregnation into a porous implant.

9. The nutritional support material according to any one of claims 1 to 7, wherein the material is in the form of chips, spheres or blocks between 2 cm and 20 cm in size.

10. The nutritional support material according to any one of claims 1 to 7, wherein the material is in the form of nanoparticles between 5 nanometers and 1 micron.

11. The nutritional support material according to any one of claims 1 to 10, further comprising an antibiotic and/or antifungal to suppress undesirable contamination growth.

12. A method of supplementing tissue survival relating to injury, disease, or surgery, comprising administering the nutritional support material of any one of claims 1 to 11 to the site of tissue injury, disease, surgery, or wound.

13. A method of supplementing autograft bone chips, the method comprising administering the nutritional support material of any one of claims 1 to 11 with a bone graft.

13. A method of supplementing cell therapy, the method comprising administering the nutritional support material of any one of claims 1 to 11 with a cell therapy.

14. A method of increasing healing of tissue injury, disease, surgery, or wound comprising introducing the nutritional support material of any of claims 1 to 11 at the site of tissue injury, disease, surgery, or wound to improve cell survival in the wound environment.

15. The method according to claim 14, wherein said material is introduced during a surgical procedure prior to closure of said site of tissue injury, disease, surgery, or wound.

16. The method according to claim 14, wherein said material is introduced to the site of tissue injury, disease, surgery, or wound prior to a surgical procedure.

17. The method according to claim 14, wherein said material is introduced to the site of tissue injury, disease, surgery, or wound subsequent to a surgical procedure after closure of said site of tissue injury, disease, surgery, or wound.

18. The method according to claim 14, wherein said material is introduced to the site of tissue injury or wound without surgical intervention.

19. The method according to any of claims 14 to 18, wherein the nutritional support material comprises a mixture of one of more of sugars, amino acids, vitamins, fatty acids, minerals, salts, and nucleic acids and a cell culture medium and has a physiologic pH.

20. The method according to any one of claims 14 to 19, wherein said method further comprises introducing waste scavenging materials at the site of tissue injury, disease, surgery, or wound to remove toxins from the site of tissue injury, disease, surgery, or wound.

21. The method according to claim 20, wherein the waste scavenging material comprises activated charcoal.

22. The method according to any one of claims 14 to 21, wherein said nutritional support material is contained within a delivery composition, optionally with said waste scavenging material.

23. The method according to claim 22, wherein said delivery composition is a solid for implant, a liquid for injection, a powder, a chip, a pellet, a microparticle, a nanoparticle, a capsule, a cream, a gel, a lotion, a paste, a coating, an impregnation of an existing implant or tissue, a combination thereof , or any biologically acceptable delivery apparatus.

24. The method according to claim 23, wherein said microparticle is comprised of one or more of PLGA or other encapsulating polymer.

25. The method according to any one of claims 14 to 24, wherein said introducing step comprises one or more of bathing, coating, layering, injecting, impregnating, or spraying the site of the tissue injury, disease, surgery, or wound with an effective amount of said support material with optional waste scavenging material.

26. The method according to any one of claims 14 to 24, wherein said introducing step comprises inserting at the site of the tissue injury, disease, surgery, or wound prior to surgical closure a delivery composition that is capable of delivering in vivo an effective amount of said support material and optional waste scavenging material.

27. The method according to any one of claims 14 to 24, wherein said introducing step comprises injecting into the site of the tissue injury, disease, surgery, or wound a delivery composition that is capable of delivering in vivo an effective amount of said support material and optional waste scavenging material.

28. The method according to claim 24, wherein said injecting occurs before, during, or after surgical closure of the site of said tissue injury or wound or performed without surgical intervention.

29. The method according to any one of claims 14-25, wherein the surgical procedure is bone grafting, tissue repair, spinal fusion, or insertion of an implant into a bone defect.

30. A composition for increasing healing at the site of a tissue injury, disease, surgery, or wound comprising an effective amount of a nutritional support material and an optional effective amount of a waste scavenging material in a physiologically acceptable delivery system.

31. A composition for implantation into the site of tissue injury, disease, surgery, or wound that is coated with a nutritional support material or a waste scavenging material.
